(19) United States
(12) Patent Application Publication
(10) Pub. No.: US 2025/0256116 A1
(43) Pub. Date: Aug. 14, 2025

(54) CARDIOPULMONARY RESUSCITATION PROTECTION DEVICE

(71) Applicants: Patrick Jack Sammons, Chicago, IL (US); Travis McPherson Sorensen, Anaheim, CA (US); Julian Mathias Andreas Blaseio, Nashville, TN (US)

(72) Inventors: Patrick Jack Sammons, Chicago, IL (US); Travis McPherson Sorensen, Anaheim, CA (US); Julian Mathias Andreas Blaseio, Nashville, TN (US)

(21) Appl. No.: 19/053,921

(22) Filed: Feb. 14, 2025

Related U.S. Application Data
(60) Provisional application No. 63/553,334, filed on Feb. 14, 2024.

(51) Int. Cl. A61N 1/39

(52) U.S. Cl. CPC A61N 1/39046; A61N 1/39044

(57) ABSTRACT

Cardiopulmonary Protection Devices may be provided by a device comprising: a chest portion having at least a first electrical resistance; a first and second wing portion, each having at least a second electrical resistance, wherein the chest portion is joined to the first wing portion and second wing portion via a first joinery section and a second joinery section, respectively; wherein the chest portion, when deployed, covers a chest area of a patient sufficient to simultaneously accommodate a rescuers hands and defibrillator pads, while at least a windpipe and neck area, a forearm area, and a subxiphoid area of the patient are unobscured by the CPR protection device; wherein the first electrical resistance is sufficient to limit a leakage current resulting from a defibrillation shock though the chest portion to a predetermined amperage.

### CROSS-REFERENCES TO RELATED APPLICATIONS

The present disclosure claims priority to U.S. Provisional Patent Application Ser. No. 63/553,334 titled “CARDIOPULMONARY RESUSCITATION PROTECTION DEVICE”, which was filed on 2024 Feb. 14, and is incorporated by reference herein in its entirety.

### BACKGROUND

Cardiopulmonary resuscitation (CPR) is part of the Advanced Cardiac Life Support (ACLS) process used to treat patients who have undergone a cardiac arrest. ACLS is aimed at sustaining the function of the body and brain with CPR and subsequently attempting to restart the heart with shocks, medication or other specific interventions if indicated. During CPR, a rescuer places their hands on the center of the chest of the subject and the rescuer repeatedly pushes on the chest, which pushes on the heart, and pumps blood to the rest of the body while the heart is not able to. While some medications and other interventions can be done in specific situations that can restart the heart, the most effective way has been shown to be defibrillation when the heart rhythm is shockable.

### SUMMARY

The present disclosure provides devices for hands-on defibrillation (HoD), which ideally prevent electrical injury to the rescuer and allow for ease of access to necessary patient contact points in order to provide advanced cardiac life support (ACLS) essential tasks.

Additional features and advantages of the disclosed method and apparatus are described in, and will be apparent from, the following Detailed Description and the Figures. The features and advantages described herein are not all-inclusive and, in particular, many additional features and advantages will be apparent to one of ordinary skill in the art in view of the figures and description. Moreover, it should be noted that the language used in the specification has been principally selected for readability and instructional purposes, and not to limit the scope of the inventive subject matter.

### BRIEF DESCRIPTION OF THE DRAWINGS

FIG. 1 illustrates a hands-on defibrillation (HoD) device, according to embodiments of the present disclosure.

FIGS. 2A-2B illustrate views of a HoD device relative to a patient, according to embodiments of the present disclosure.

FIGS. 3A-3C illustrate various example joinery sections of a HoD device, according to embodiments of the present disclosure.

FIGS. 4A-4B illustrate various example folded configurations of HoD devices, according to embodiments of the present disclosure.

FIGS. 5A-5B illustrate various example rolled configurations of HoD devices, according to embodiments of the present disclosure.

### DETAILED DESCRIPTION

The present disclosure provides devices for hands-on defibrillation (HoD), in which a patient based electrical barrier is provided. HoD is a technique used to reduce the duration of peri-shock pause during cardiopulmonary resuscitation (CPR). Peri-shock pause is a necessary pause that takes place during CPR when a rescuer stops performing chest compressions in order to check pulses and, if indicated, administer a defibrillating shock before resuming chest compressions. Often, patients will require multiple defibrillation shocks in order to be successfully resuscitated, and the duration of the peri-shock pauses is inversely correlated with survival rates of patients. Studies indicate that reducing the duration of peri-shock pause to less than twenty seconds increases survival rates of patients by about 11%.

In typical CPR procedures, a rescuer cannot be in contact with the patient during a defibrillating shock, as electrical leakage from the defibrillator can be transmitted to the rescuer, resulting in electrical shock, and potentially cardiac arrest for the rescuer. In order to prevent such an undesirable outcome, certain preventative practices have been deployed. The majority of these preventative practices involve some form of electrical barrier that provides the rescuer with protection against electrical leakage. Implements that provide an electrical barrier can be considered in two categories: rescuer based barriers and patient based barriers. Most early trials focused on rescuer based barriers with readily available materials in the hospital. All types of single or double layer hospital gloves failed to adhere to International Electrotechnical Commission (IEC) safety standards following a brief period of normal use. Only two modalities (electrical safety gloves, firefighter gloves) have thus far met IEC safety standards.

While there are at least two rescuer based modalities that work, the strategy of using gloves as barriers is impractical due to the number of rescuers performing chest compressions. Individuals performing compressions may experience fatigue, and multiple rescuers may perform compressions in a single cardiac arrest, necessitating many gloves for different rescuers. Furthermore, there is a risk of an inadvertent secondary connection to the patient, for example, as a rescuer is leaning over the patient performing compressions, the rescuer's leg may contact the patient's shoulder, creating a path of conduction for electrical leakage.

Peer-reviewed experimentation involving patient based barriers has been minimal, but has been substantially limited to the implementation of a 3-foot by 3-foot (3×3) sheet of 0.05 millimeter (mm) thick polyethylene draped over the patient. Experimentation showed that the barrier meets IEC safety standards at the secondary point of contact (arm), but not at the primary contact point (chest). The differences in safety standards have been correlated with distance from the defibrillator pads, as the human body has an innate electrical resistance, and the secondary contact points, being further from the pads, experience lower current leakage than the chest area, being immediately proximate to the pads. Further failings are also present in the experimentation. In the case of most patients, the 3×3 sheet covers the majority of the head, face, torso, shoulders, arms, abdomen and pelvis. This obstruction restricts access to the arms, which are necessary intravenous (IV) access points, access to the head and neck, which is necessary to ensure a patent airway and adequate breaths, and access to other areas of the patient necessary to perform the essential tasks of advanced cardiac life support (ACLS) resuscitation.

The devices and apparatuses of the present disclosure are intended to remedy certain aforementioned deficiencies of the 3×3 polyethylene sheet as a patient based barrier for HoD.

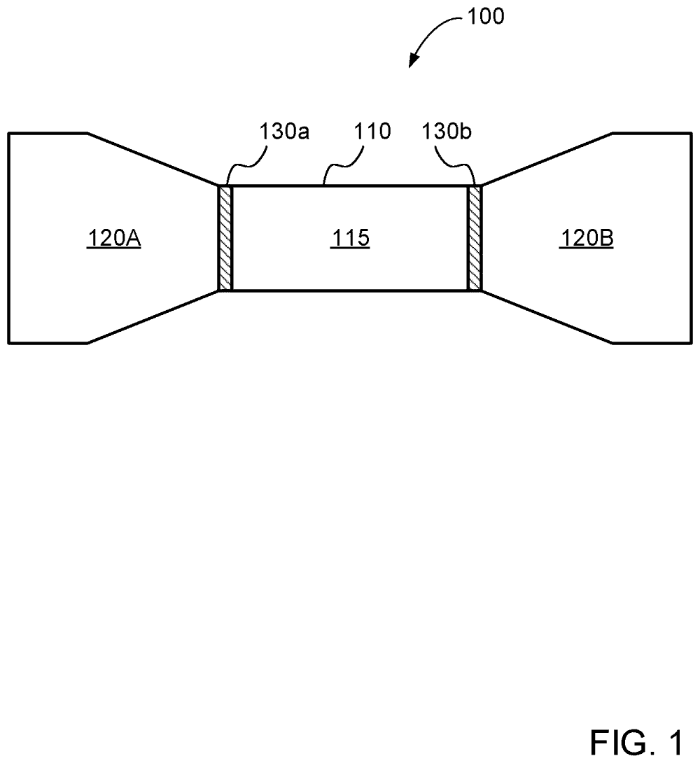

FIG. 1 illustrates a HoD device 100, (e.g., a CPR protection device, apparatus) including a chest portion 110 defining a defibrillation area 115, and a first wing portion 120a, a second wing portion 120b (generally or collectively, wing portions 120), and the wing portions 120 are joined to the chest portion 110 by joinery sections 130a and 130b (generally or collectively, joinery sections 130), according to embodiments of the present disclosure.

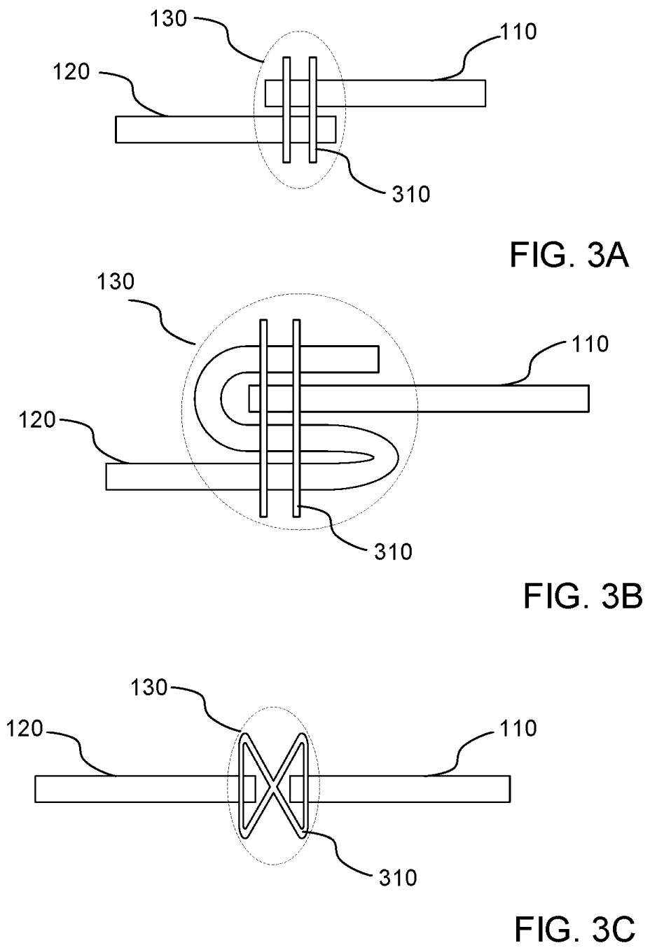

The chest portion 110 is an electrical protection means, and defines the defibrillation area 115, and is configured to facilitate the delivery of defibrillation shocks to a patient, while substantially decreasing the risk of electric shock via current leakage to the rescuer administering CPR to a patient. The wing portions 120 may be laterally joined to the chest portion 110 by one or more of several methods of joinery (as discussed in greater detail in regard to FIGS. 3A-3C), and the wing portions 120 are configured to provide a patient-rescuer barrier at the patient's shoulders and arms, which are frequent points of incidental secondary contact in conventional designs. The secondary contact point barrier provided by the wing portions 120 reduces the risk of electric shock via current leakage to the rescuer. A shape of the profile of the HoD device 100 possesses reflection symmetry along a lateral axis bisecting each of the wing portions 120, each of the joinery sections 130 and the chest portion 110, as well as reflection symmetry along a longitudinal axis perpendicular to the lateral axis, and bisecting the chest portion 110.

In order to comply with IEC 60601-1 Standard for Medical Electrical Equipment for leakage current (less than 0.5 milliamps (mA)), the chest portion 110 is constructed of at least a first material with a minimum electrical resistance of 10 mega-Ohms (MΩ). The wing portions 120 are constructed of at least a second material with a minimum electrical resistance of 5 MΩ. In certain examples, both the chest portion 110 and wing portions 120 may be constructed of the same material, so long as the material satisfies the 10 MΩ minimum resistance requirement for the chest portion 110. The minimum resistance is determined in order to limit the leakage current below a predetermined amperage (e.g., the IEC 60601-1 Standard of 0.5 mA).

The chest portion 110 is substantially rectangular, and the shape of the wing portions 120 may vary between embodiments, including wing portions 120 that are substantially rectangular, substantially trapezoidal, substantially circular, substantially ovular, and substantially semi-circular or substantially an irregular polygonal shape. The chest portion 110 is sized such that a rescuer performing CPR may easily and consistently perform accurate chest compressions on a patient without incurring an incidental contact with the patient outside of the barrier provided by the HoD device 100. The wing portions 120 are generally larger in surface area than the chest portion 110, allowing for rescuers to kneel or lean thereupon when the HoD device 100 is draped over patients of various sizes. The form of the HoD device 100 is symmetrical and bi-directional, such that application of the HoD device is not encumbered by attempts to re-orient the HoD Device 100 prior to deployment. Moreover, the bi-directionality allows a second rescuer to arrive and begin chest compressions from the opposite side of the patient to the first rescuer without requiring the first rescuer to stand up and yield the position, thereby avoiding or minimizing undesirable pauses in chest compressions when switching between rescuers.

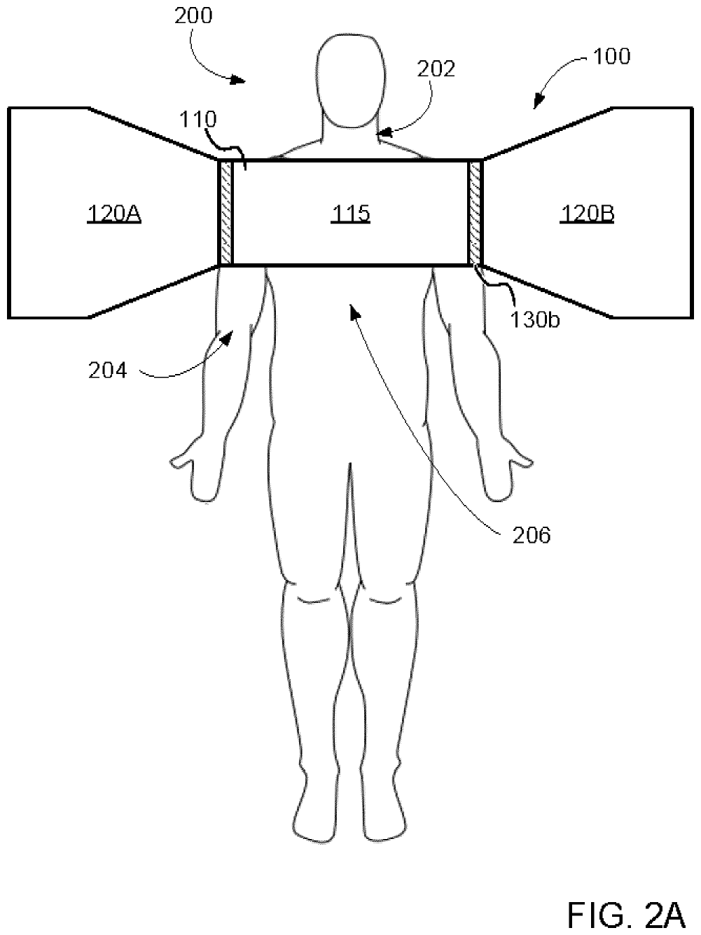

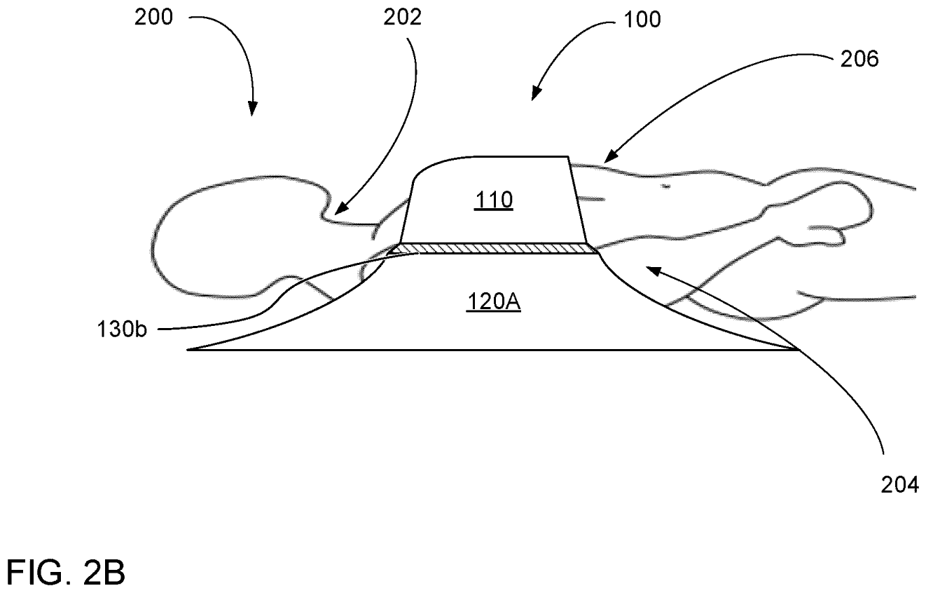

FIGS. 2A and 2B illustrate a HoD device 100 draped over a patient 200 from different perspectives, according to embodiments of the present disclosure. The configuration of the HoD device 100 results in a series of advantages over the previously implemented square polyethylene sheets. The chest portion 110 is sized such that, when applied to a patient 200 of typical size and proportions, the head, neck area 202, abdomen, and pelvis of the patient are not obscured by the HoD device 100; yet the chest portion 110 is of sufficient size that a rescuer may perform chest compressions on the patient without the rescuer's hands directly contacting the patient.

By leaving the neck and windpipe area 202, antecubital and forearm area 204, and subxiphoid area 206 exposed, the HoD device 100 allows for access to the patient in the areas necessary to perform the ACLS essential tasks. Access to the head and neck area 202 is necessary for checking pulse, central line placement, performing bag valve mask ventilation and obtaining a definitive airway such as laryngeal airway placement or endotracheal intubation. Access to the antecubital and forearm area 204 is necessary for IV insertion, and access to the subxiphoid area 206 is necessary for lung, cardiac and pericardial ultrasounds.

According to some embodiments, the chest portion 110 is form fitting, e.g., the chest portion possesses elastic and tactile properties such that the HoD device 100 may be draped on the patient in a manner that reduces slipping and crumpling of the chest portion. In some examples, the chest portion 110 is constructed of a material having a natural tackiness, which is conducive to a temporary static adhesion of the HoD Device 100 to the patient 200.

In various embodiments, one or both faces of the chest portion 110 may include grip-increasing sections for reducing slipping and other inadvertent movement of the HoD Device 100 relative to the patient 200 after the device has been deployed and CPR has been initiated. Grip-increasing sections may include materials with an inherent or manufactured tackiness or substantial static cling, and in some examples include applied adhesives. According to some embodiments, grip-increasing sections include an applied adhesive with a liner temporarily adhered to the adhesive, the liner to be removed prior to deployment such that the adhesive facilitates the adherence of the HoD Device 100 to the garments or person of the patient 200. In some examples, the grip-increasing sections may be located proximately to, or directly on, the joinery sections 130.

According to some embodiments, the chest portion 110 may be constructed of materials such as silicone, polyvinyl chloride, polypropylene, polyethylene, acrylics, nylon, polycarbonate, plastics, bamboo derivatives and combinations thereof.

Furthermore, the wing portions 120 may be constructed to have sufficient size that two rescuers may kneel on opposing wing portions 120, and apply compression to the patient's chest by introducing tension in the chest portion 110 by applying simply bodyweight to the opposing wing portions 120. In such examples, the joinery sections 130 are of sufficient strength to withstand the tensile force applied to the HoD device 100. According to some embodiments, the wing portions 120 may be constructed of materials such as silicone, polyvinyl chloride, polypropylene, polyethylene, acrylics, nylon, polycarbonate, plastics, bamboo derivatives and combinations thereof. In various embodiments, the joinery sections 130 are constructed of a material having a natural tackiness, which is conducive to a temporary static adhesion of the HoD device 100 to the patient 200.

FIG. 3A-3C illustrate various joints formed by the joinery sections 130 between the chest portion 110 and a wing portion 120, according to embodiments of the present disclosure. According to some embodiments, the joinery sections 130 are primarily made of overlapping material from the chest portion 110 and wing portions 120. The overlapping material from the chest portion 110 and wing portions 120 may be secured by one of a number of methods of joinery, including but not limited to stitching, heat bonding, and adhering via adhesive. Joinery by stitching may include stiches through multiple folds of overlapping material to increase the strength of the joinery section 130. Stitches may include two fold stitches, double stitches, bar tack stitches, diamond stitches, and W-stitches. Heat bonding may be used as a form of joinery in examples where the chest portion 110 or wing portions 120 are constructed with a compatible thermoplastic material. Various adhesives may be used to adhere the overlapping material from the chest portion 110 and wing portions 120, such as glues, acrylates and thermoplastic bonding agents.

FIG. 3A illustrates a cross-sectional view of a double stitch joinery section 130 with threads 310 passing though overlapping portions of the chest portion 110 and wing portion 120.

FIG. 3B illustrates a cross-sectional view of a two-fold stitch joinery section 130, with the chest portion 110 inserted into a Z-fold of the wing portion 120 and threads 310 passing through the chest portion 110 once, and the wing portion 120 three times.

FIG. 3C illustrates a cross-sectional view of a baseball stitch joinery section 130, with threads 310 joining the chest portion 110 to an adjacent wing portion 120.

In various embodiments, the joinery section 130 may alter the joining means at various portions along the length of the joint between the chest portion 110 and the wing portion 120 to provide greater or lesser joining force at portion of the HOD device 100 at certain mechanical stress points. Additionally, or alternatively, the first joinery section 130a may use the same or a different joinery technique than the second joinery section 130b. Accordingly, the present disclosure contemplates that any of the illustrated examples shown in FIGS. 3A-3C may be included in a single embodiment.

In some examples, the HoD device 100 may include sections having an applied adhesive. The HoD device may be supplied to the end user with liners disposed on the adhesive portions, the liners being removed prior to deployment on a patient, thus revealing the adhesive, and facilitating adhesion to the patient.

According to some embodiments, the HoD device 100 may include printed media on one or both faces of one or all of the chest portion 110 and wing portions 120, such as a schematic, to aid in a rescuer's ability to properly place the HoD device 100 and perform CPR. A non-limiting example schematic may include a diagram showing the location of the heart, lungs, or other anatomy, and include orienting symbols such as arrows or phrases indicating ideal placement of the device on a patient, or CPR instructions. The present disclosure contemplates that the printed media may include symbols, diagrams, schematics, natural language text, images, and combinations thereof.

According to some embodiments, the HoD device 100, or a portion thereof, is visually transparent. Transparency allows a rescuer to more accurately place the HoD device 100, as the patient's anatomy is viewable through the HoD device 100, facilitating increased alignment precision. In various embodiments, the chest portion 110 is more transparent than the wing portions 120; allowing greater visibility through the chest portion 110.

According to some embodiments, the HoD device 100 is fire-resistant. Although the HoD device 100 is not expected to encounter open flames in course of intended use, the high voltage of defibrillator shocks presents a flame risk. In some examples, the HoD device 100 is constructed from flame-resistant materials. In some examples the HoD device 100 includes a flame-retardant coating, layer, portion, or other element intended for preventing the HoD device 100 from catching fire.

In some examples, the HoD device 100 is recyclable or otherwise bio-disposable. As the HoD device 100 is intended to be a single-use device (e.g., for sterility reasons), disposability is a motivating concern. In certain embodiments, the materials chosen for the HoD device 100 may be readily recyclable, biodegradable, or bio-erodible materials. Furthermore, as a single use device, high manufacturing costs per unit are undesirable, and the materials may also be chosen with cost in mind.

According to some embodiments, the HoD device 100 arrives to the end user in a compacted, rolled, or folded form, which may include other elements used for rapid transitions from compact storage to deployment, such as strings, straps, hook and loop fasteners, and adhesives.

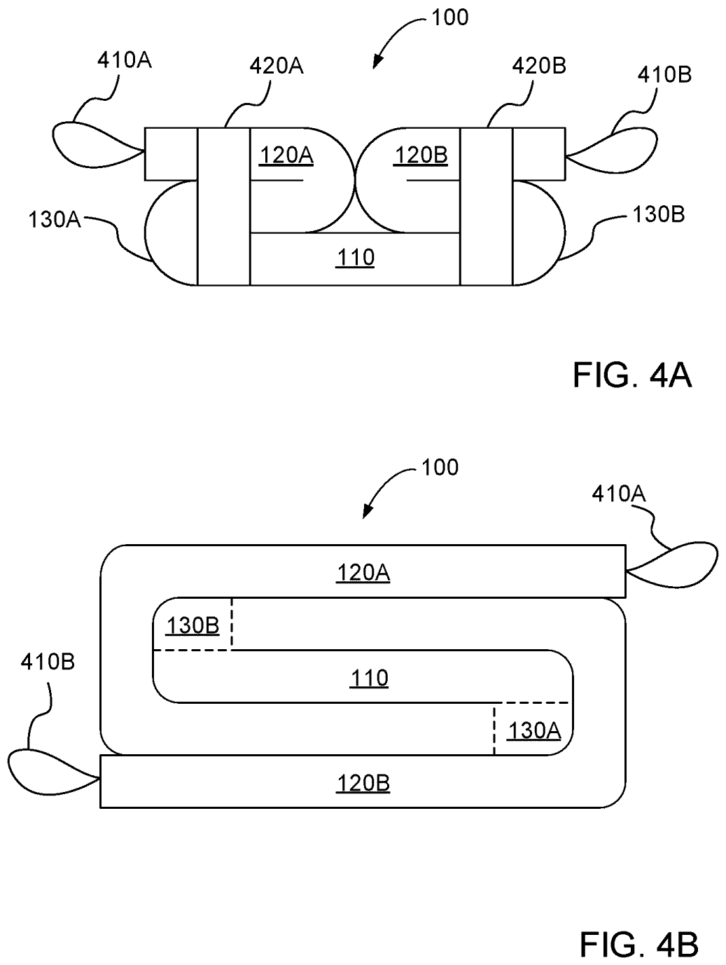

FIGS. 4A-4B illustrate various folded configuration using trailers 410a-b (collectively trailers 410) of string, twine, or rope integrated with the wing portions 120 or the material used to construct the wing portions 120 or a strap 420 separately defined from the HoD device 100.

FIG. 4A illustrates a first folded configuration of HoD device 100. Each wing portion 120 is accordion folded so as to fit in a space defined by half of the chest portion 110. Each folded wing portion 120 is secured by a strap 420 (420a and 420b, respectively) which may be implemented by simple tension, hook and loop fastening, or adhesives. The trailers 410 may be in the form of pull tabs, string, twine, rope, cord, or handle attached to the outermost wing portions 120. In some embodiments, the straps 420 are configured such pulling both trailers 410a-b frees the HoD device 100 from the straps and extends the accordion folds, such that the HoD device 100 is in condition for deployment. In other configurations, the straps 420 must be removed entirely before the trailers 410 may be effectively pulled to extend the accordion folds. Although illustrated as a single accordion fold of each wing portion 120, this disclosure contemplates that the wing portions 120 may be folded many times to accommodate various size constraints.

FIG. 4B illustrates a second folded configuration of HoD device 100. The wing portions 120 are folded in a spiraled manner about the chest portion 110. Much like the first folded configuration, the second folded configuration may be unfurled by pulling the trailers 410, which expands the HoD device 100 for deployment. In some examples, the second folded configuration includes a strap(s) 420, disposed longitudinally about the folded HoD device 100 or about a circumference thereof. Although illustrated as a single spiral fold of each wing portion 120, this disclosure contemplates that the wing portions 120 may be folded many times to accommodate various size constraints.

Additionally, although illustrated with an unfolded chest portion 110 for clarity in illustration, the present disclosure contemplates that the chest portion 110 may also be folded one or more times to thereby accommodate various size constraints for the storage or shipment of the HoD device 100.

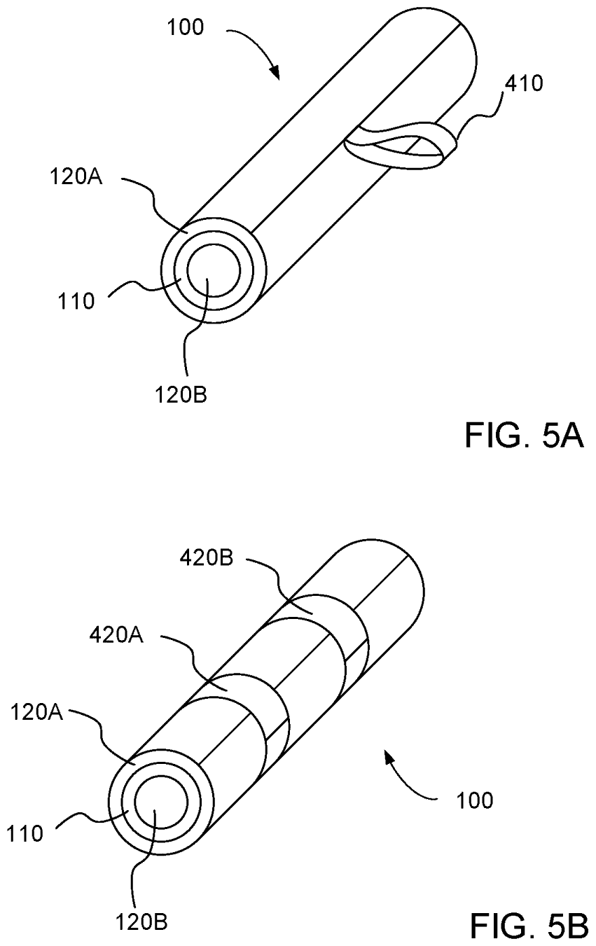

FIGS. 5A-5B illustrate various rolled arrangements, which may include trailers 410 (including pull tabs, string, twine, rope, cord or wires), adhesives, and straps 420, integrated with an outermost wing portions 120b or the material used to construct the outermost wing portions 120b. In some embodiments, various HoD devices 100 in rolled arrangements may include constructional differences relative to HoD devices 100 in folded arrangements, such as, but not limited to, the locations trailer 410 or strap 420 attachment points, and number of straps 420 and trailers 410. In other embodiments, a HoD device 100 may be configured such that both a rolled configuration and folded configuration are possible and practical, and a user may select the configuration according to preference, without necessitating the selection of a separate HoD device 100. Furthermore, this disclosure contemplates various folded and rolled configurations of HoD devices 100 that do not appear in the illustrated embodiments, including configurations implementing both folded and rolled sections.

Certain terms are used throughout the description and claims to refer to particular features or components. As one skilled in the art will appreciate, different persons may refer to the same feature or component by different names. This document does not intend to distinguish between components or features that differ in name but not function.

As used herein, “about,” “approximately” and “substantially” are understood to refer to numbers in a range of the referenced number, for example the range of −10% to +10% of the referenced number, preferably −5% to +5% of the referenced number, more preferably −1% to +1% of the referenced number, most preferably −0.1% to +0.1% of the referenced number.

Furthermore, all numerical ranges herein should be understood to include all integers, whole numbers, or fractions, within the range. Moreover, these numerical ranges should be construed as providing support for a claim directed to any number or subset of numbers in that range. For example, a disclosure of from 1 to 10 should be construed as supporting a range of from 1 to 8, from 3 to 7, from 1 to 9, from 3.6 to 4.6, from 3.5 to 9.9, and so forth.

As used in the present disclosure, a phrase referring to “at least one of” a list of items refers to any set of those items, including sets with a single member, and every potential combination thereof. For example, when referencing “at least one of A, B, or C” or “at least one of A, B, and C”, the phrase is intended to cover the sets of: A, B, C, A-B, B-C, A-C, and A-B-C, where the sets may include one or multiple instances of a given member (e.g., A-A, A-A-A, A-A-B, A-A-B-B-C-C-C, etc.) and any ordering thereof. For avoidance of doubt, the phrase “at least one of A, B, and C” shall not be interpreted to mean “at least one of A, at least one of B, and at least one of C”.

Without further elaboration, it is believed that one skilled in the art can use the preceding description to use the claimed inventions to their fullest extent. The examples and aspects disclosed herein are to be construed as merely illustrative and not a limitation of the scope of the present disclosure in any way. It will be apparent to those having skill in the art that changes may be made to the details of the above-described examples without departing from the underlying principles discussed. In other words, various modifications and improvements of the examples specifically disclosed in the description above are within the scope of the appended claims. For instance, any suitable combination of features of the various examples described is contemplated.

Within the claims, reference to an element in the singular is not intended to mean “one and only one” unless specifically stated as such, but rather as “one or more” or “at least one”. Unless specifically stated otherwise, the term “some” refers to one or more. No claim element is to be construed under the provision of 35 U.S.C. § 112(f) unless the element is expressly recited using the phrase “means for” or “step for”. All structural and functional equivalents to the elements of the various embodiments described in the present disclosure that are known or come later to be known to those of ordinary skill in the relevant art are expressly incorporated herein by reference and are intended to be encompassed by the claims. Moreover, nothing disclosed in the present disclosure is intended to be dedicated to the public regardless of whether such disclosure is explicitly recited in the claims.

The invention is claimed as follows:

1. A cardiopulmonary resuscitation (CPR) protection device comprising:
a chest portion having at least a first electrical resistance;
a first wing portion and a second wing portion, each having at least a second electrical resistance;
wherein the chest portion is joined to the first wing portion and second wing portion via a first joinery section and a second joinery section, respectively;
wherein the chest portion, when deployed, covers a chest area of a patient sufficient to accommodate a rescuers hands, while at least a head and neck area, a forearm and antecubital area, and a subxiphoid area of the patient are readily accessible or unobscured by the CPR protection device;
wherein the first electrical resistance is sufficient to limit a leakage current resulting from a defibrillation shock though the chest portion to a predetermined amperage; and
wherein the second electrical resistance is sufficient to limit a leakage current resulting from a defibrillation shock though the first wing portion or the second wing portion to the predetermined amperage.

2. The CPR protection device of claim 1, wherein the first electrical resistance is 10 mega-Ohms (MΩ).

3. The CPR protection device of claim 2, wherein the second electrical resistance is 5 MOhm.

4. The CPR protection device of claim 1, wherein the predetermined amperage is at most 0.5 milliamps (mA).

5. The CPR protection device of claim 1, wherein the first joinery section and the second joinery section comprise one or more forms of joinery selected from a group consisting of a two-fold stitch, a double stitch, bar tack stitch, diamond stitch, w-stitch, a heat weld, glue, and combinations thereof.

6. The CPR protection device of claim 1, wherein the chest portion is constructed from material selected from a group consisting of silicone, polyvinyl chloride, polypropylene, polyethylene, acrylics, nylon, polycarbonate, plastics, bamboo derivatives and combinations thereof.

7. The CPR protection device of claim 1, wherein the first and second wing portions are constructed from material selected from a group consisting of silicone, polyvinyl chloride, polypropylene, polyethylene, acrylics, nylon, polycarbonate, plastics, bamboo derivatives, and combinations thereof.

8. The CPR protection device of claim 1, further comprising printed media selected from a group consisting of symbols, diagrams, schematics, natural language text, images, and combinations thereof.

9. The CPR protection device of claim 1, wherein the chest portion is visually transparent.

10. An apparatus, comprising:
an electrical protection means having a rectangular cross section of a given height and of a given length;
a first wing connected to the electrical protection means on a first end thereof to a first side of the electrical protections means, the first wing having a cross section with the given height on the first end and a second height, greater than the given height, on a second end opposite to the first end; and
a second wing connected to the electrical protection means on a first end thereof to a second side of the electrical protections means, opposite to the first side, the second wing having a cross section with the given height on the first end and the second height on a second end opposite to the first end.

11. The apparatus of claim 10, wherein one or more of the electrical protection means, the first wing, and the second wing, is visually transparent.

12. The apparatus of claim 11, wherein the electrical protection means has a greater visual transparency than that of the first wing and the second wing.

13. The apparatus of claim 10, wherein the electrical protection means has a thickness greater than that of the first wing and the second wing.

14. The apparatus of claim 10, wherein the electrical protection means is constructed of a material selected from a group consisting of silicone, polyvinyl chloride, polypropylene, polyethylene, acrylics, nylon, polycarbonate, plastics, bamboo derivative, and combinations thereof.

15. The apparatus of claim 10, wherein the first wing and the second wing are constructed of a material selected from a group consisting of silicone, polyvinyl chloride, polypropylene, polyethylene, acrylics, nylon, polycarbonate, plastics, and bamboo derivatives, and combinations thereof.

16. The apparatus of claim 10, wherein the electrical protection means is attached to the first wing and the second wing by at least one of stitching, heat bonding, and adhesive.

17. The apparatus of claim 10, wherein a length of the first wing and the second wing is at least twice the length of the electrical protection means.

18. The apparatus of claim 10 wherein in an undeployed state, the apparatus is compacted by a process chosen from a group consisting of rolling, over/under folding, booklet folding, accordion folding, and combinations thereof.

19. The apparatus of claim 18, further comprising a securing means, selected from a group consisting of string, cord, twine, rope, and straps.

20. The apparatus of claim 10, wherein a shape of a profile of the apparatus possesses reflection symmetry along a longitudinal axis and a central axis perpendicular to the longitudinal axis.
